(19) United States
(12) Patent Application Publication
(10) Pub. No.: US 2006/0024263 A1
(43) Pub. Date: Feb. 2, 2006

(54) Polymer compositions

(76) Inventors: Steven Van Es, Liege (BE); Olivier Dupont, Chastre (BE); Willy Segers, Jurbise (BE)

Correspondence Address: WENDEROTH, LIND & PONACK, L.L.P., 2033 K STREET N. W., SUITE 800, WASHINGTON, DC, 20006-1021, US

(21) Appl. No.: 10/533,943

(22) PCT Filed: Dec. 1, 2003
(86) PCT No.: PCT/EP03/13496

§ 371 Date: Jun. 8, 2005

(30) Foreign Application Priority Data
Dec. 6, 2002 (EP) 02027263.9

(51) Int. Cl. A61K 8/81; C08F 120/02

(52) U.S. Cl. 424/70.16; 525/329.7

(57) ABSTRACT

There is disclosed polymers and aqueous dispersions of them, the polymers containing specific copolymers which comprise at least four different ethylenically unsaturated monomers (such as 2-ethylhexyl acrylate, ethyl acrylate, methyl acrylate and styrene, and optionally (meth)acrylic acid, ethyl imidazolidone methacrylate, n-butyl acrylate n-dodecyl methacrylate and/or methyl methacrylate). Also described is production of said polymers and dispersions by emulsion polymerization and their use as adhesives, for example as pressure sensitive adhesives on polymeric film.

The invention relates to polymers, aqueous dispersions comprising said polymers, to their use for example as adhesives and to the preparation thereof.

Pressure sensitive adhesives (PSA) form a permanently adhesive film capable of adhering to various surfaces upon slight pressure at ambient temperature. Pressure sensitive adhesives serve for the manufacture of self-adhesive products, such as labels, tapes or films. Such products are easy to handle and allow fast processing. In contrast to conventional adhesives, no curing nor solvent evaporation is required.

The quality of a substrate applied with a self-adhesive film is usually dependent on the adjustment of the internal strength within the material (cohesion) and the external affinity to the substrate (adhesion). Particularly for pressure sensitive adhesives used to produce self-adhesive films, tapes or labels, a high degree of cohesion, i.e. shear strength, is required, as under the conditions of further processing of these products (cutting, stamping, etc.) the adhesive film should firmly remain on the substrate. At the same time, the adhesion, i.e. the peel strength and loop tack of the product should meet the requirements for the intended application. Simultaneously, adhesion and cohesion of a pressure sensitive adhesive are difficult to adjust.

Besides good adhesion and good cohesion there are further properties of pressure sensitive adhesives which may be desired. Yet many pressure sensitive adhesives when applied to a transparent substrate may undesirably become whiter and more opaque in the presence of water or high humidity. This water whitening or blush is to be avoided for many uses (such as no-look labels) where the appearance of the substrate is important.

U.S. Pat. No. 6,359,092 discloses an aqueous, blush-retardant pressure sensitive adhesive which comprises an aqueous latex emulsion emulsified in the presence of the aromatic emulsifier poly(oxy-1,2-ethanediyl), α-sulfo-α-[4-nonyl-2-(1-propenyl)-pheny-oxyl]branched ammonium salt (Hitenol BC-10).

JP 2002-053819 (Mitsui Chem) describes a readily peelable, radiation cured adhesive film which is applied to a semiconductor wafer as a protective water barrier.

JP 8027450 (Mitsui Toatsu Chem) describes a copolymer C1-12 alkyl ester methacrylate for use in PSA compositions.

JP 7003221 (Sekisui Chem) describes a decorative film coated with PSA for applying to glass where the PSA comprises an acrylic resin and a silane coupling agent.

It is an object of the invention to provide aqueous polymer dispersions, which are suitable as adhesives.

It has been surprisingly found that this underlying technical problem is solved by the subject matter of the patent claims.

The invention is based on the unexpected finding that certain aqueous polymer dispersions are advantageous adhesives, which do not show the water-whitening effect. The polymer dispersions contain polymers, which are abbreviated as P in the present application. It was also found that polymers falling within the above definitions, which have a specific structure, which is caused by a specific production process, are particularly suitable for carrying out the invention.

Broadly in accordance with the invention there is provided a polymer (Polymer P) which comprises constituents ‘a’, ‘b’, ‘c’ and ‘d’, optionally together with constituents ‘e’ and/or ‘f’, where each of these constituents are different from each other.

In accordance with another aspect of the invention there is provided an aqueous dispersion comprising Polymer P.

Polymer P comprises (preferably consists essentially of), is obtained and/or is obtainable from the following monomers:
(a) from about 5 to about 92.5 wt.-% of at least one alkyl acrylate, the homopolymers of which have a glass transition temperature (Tg) of less than or equal to about −40° C. (constituent ‘a’);
(b) from about 2.5 to about 30 wt.-% of at least one alkyl (meth)acrylate the homopolymers of which have Tg of from about −25° C. to about 0° C. (constituent ‘b’;
(c) from about 2.5 to about 30 wt.-% of at least one alkyl (meth)acrylate the homopolymers of which have a glass transition temperature of from about 0° C. to about 20° C. (constituent ‘c’);
(d) from about 1 to about 30 wt.-% of at least one ethylenically unsaturated monomer the homopolymers of which have a Tg of greater than or equal to about 20° C. and which comprise functional groups other than any selected from hydroxyl, acid, acid anhydride, nitro, epoxy and amino groups (constituent ‘d’),
(e) from about 0 to about 10 wt.-% of at least one ethylenically unsaturated monomer having at least one acid group or acid anhydride group (constituent ‘e’), and
(f) from about 0 to about 60 wt.-% of at least one ethylenically unsaturated monomer having either (i) no further functional group or (ii) in addition to the ethylenically unsaturated group at least one functional group other than an acid group or an acid anhydride group (constituent ‘f’).

All values in wt.-% are based on the total weight of the polymer.

From the above definition it follows that Polymer P comprises constituents ‘a’, ‘b’, ‘c’ and ‘d’. As the content of constituent(s) ‘e’ and/or ‘f’ may be 0 wt.-%, the presence of these two constituent(s) are only optional.

In this specification the term “(meth)acryl” means “acryl” as well as “methacryl”.

Unless the context clearly indicates otherwise, as used herein plural forms of the terms herein are to be construed as including the singular form and vice versa.

The term “comprising” as used herein will be understood to mean that the list following is non-exhaustive and may or may not include any other additional suitable items, for example one or more further feature(s), component(s), ingredient(s) and/or substituent(s) as appropriate. In contrast the term “consisting essentially of” as used herein will be understood to mean that the list following is exhaustive and does not include any additional items.

Compounds which are usually not interpreted as polymerizable monomers, such as initiators or chain transfer agents, are preferably not to be understood as constituents of Polymer P according to the invention. Thus, a polymer which comprises constituents ‘a’, ‘b’, ‘c’, ‘d’, ‘e’ and ‘f’ may also comprise, for example, fragments of radical initiators and/or chain transfer agents covalently bonded to the polymer. Preferably Polymer P does not comprise such additional ingredients.

Constituent ‘a’

Preferably the monomer(s) for constituent ‘a’ comprise: C1-C20alkyl acrylates the homopolymers of which have a Tg of less than or equal to about −40° C., more preferably C1-C10 alkyl acrylates, most preferably selected from butyl acrylate, 2-ethylbutyl acrylate, hexyl acrylate, 2-ethylhexyl acrylate, nonyl acrylate and n-octyl acrylate. The monomers n-butyl acrylate and 2-ethylhexyl acrylate are particularly preferred, especially 2-ethylhexyl acrylate. When Polymer P comprises 2-ethylhexyl acrylate it is preferred that n-butyl acrylate is present in only minor amounts, preferably less than 5 wt.-%, more preferably less than 2 wt.-%, in particular less than 1 wt-%, for example 0 wt-% of Polymer P.

Conveniently constituent ‘a’ comprises from about 40 to about 90 wt.-%, more conveniently from about 55 to about 80 wt.-%, most conveniently from about 60 to about 70 wt.-% of the total weight of Polymer P.

Constituent ‘b’

Preferably the monomer(s) for constituent ‘b’ comprise: C1-C20alkyl (meth)acrylates the homopolymers of which have a Tg of from about −25° C. to about 0° C. (usefully from about −25° C. to about −5° C.), more preferably C1-C12alkyl (meth)acrylates. Alkyl acrylates are most preferred over the corresponding alkyl methacrylates. Ethyl acrylate, sec-butyl acrylate, isobutyl acrylate and isopropyl acrylate are preferred in particular, especially ethyl acrylate.

Conveniently constituent ‘b’ comprises from about 5 to about 30 wt.-%, more conveniently from about 8 to about 20 wt.-%, most conveniently from about 10 to about 15 wt.-% of the total weight of Polymer P.

Constituent ‘c’

Preferably the monomer(s) for constituent ‘c’ comprise: C1-C20 alkyl (meth)acrylates the homopolymers of which have a Tg of from about 0° C. to about 20° C. (useflllly from about 5° C. to about 20° C.), more preferably C1-C12 alkyl (meth)acrylates. Methyl acrylate and n-butyl methacrylate are most preferred, particularly methyl acrylate.

Conveniently constituent ‘c’ comprises from about 5 to about 30 wt.-%, more conveniently from about 8 to about 20 wt.-%, most conveniently from about 10 to about 15 wt.-% of the total weight of Polymer P.

Desirably the total amount of constituents ‘b’ and ‘c’ comprising Polymer P is from about 5 to about 30 wt.-%, more desirably from about 15 to about 30 wt.-%, most desirably from about 22 to about 26 wt.-%. of the total weight of Polymer P In particular it is desired that polymer P comprises equal amounts of constituents ‘b’ and ‘c’.

Constituent ‘d’

Preferably the monomer(s) for constituent ‘d’ are selected from the group consisting of:
(i) alkyl (meth)acrylates, more preferably C1-C20alkyl (meth)acrylates the homopolymers of which have a Tg of greater than or equal to about 20° C. (usefully greater than or equal to about 25° C.), most preferably C1-C10alkyl (meth)acrylates, in particular methyl methacrylate and tert-butyl acrylate, especially methyl methacrylate;
(ii) vinylaromatic compounds, more preferably C8-C20 vinylaromatic compounds, the homopolymers of which have a Tg of greater than or equal to about 20° C., most preferably C8-C20 styrene derivatives, in particular metylstyrene, para-methylstyrene, 4-tert-butylstyrene, 4-sec-butylstyrene and styrene, especially styrene;
(iii) vinylhalides, more preferably ethylenically unsaturated compounds substituted with chlorine, fluorine or bromine the homopolymers of which have a Tg of greater than or equal to about 20° C., most preferably C2-C5 vinylhalides, in particular vinyl chloride;
(iv) vinylnitriles, more preferably C2-C5vinylnitriles the homopolymers of which have a Tg of greater than or equal to about 20° C., most preferably methacrylonitrile and acrylonitrile; and
(v) vinylesters of carboxylic acids, more preferably vinylesters of C1-C20 carboxylic acids the homopolymers of which have a Tg of greater than or equal to about 20° C., most preferably vinyl acetate and vinyl formate.

Conveniently constituent ‘d’ comprises from about 1 to about 15 wt.-%, more conveniently from about 1 to about 5 wt.-%, most conveniently from about 2 to about 4 wt.-% of the total weight of Polymer P.

Constituent ‘e’

Preferably the monomer(s) for constituent ‘e’ comprise: ethylenically unsaturated compounds comprising carboxyl groups, (such as (meth)acrylic and/or maleic acids) ethylenically unsaturated acid anhydrides or monoesters of ethylenically unsaturated diacids or triacids (such as maleic acid anhydride, maleic acid monoester and/or fumaric acid monoester). Acrylic acid and methacrylic acid are more preferred, particularly a mixture thereof.

Conveniently constituent ‘e’ comprises from about 0.5 to about 6 wt.-%, more conveniently from about 0.5 to about 5 wt.-%, most conveniently from about 3.5 to about 5 wt.-% of the total weight of Polymer P.

Constituent ‘f’

Preferably the monomer(s) for constituent ‘f’ comprise those ethylenically unsaturated compounds which customarily enhance the internal strength and/or adhesion of the films formed from an aqueous polymer dispersion comprising Polymer P.

In addition to the ethylenically unsaturated group these compounds may comprise either no further functional group or one or more further functional group(s) preferably selected from epoxy, hydroxyl, ethyl imidazolidone, N-methylol, carbonyl and/or a further ethylenically unsaturated group which is not conjugated with the other ethylenically unsaturated group. The further functional group(s) may only comprise an acid or an acid anhydride group if additionally one or more additional further functional groups other than the acid or an acid anhydride group are also present in the molecule.

More preferred further functional group(s) are selected from. —OR, —OOR, —SR, —SSR, —COR, —COHal, —CO2R, —OCO2R, —OCONRR′, —NRCO2R′, —CONRR′, —NRCONR′R″, —NRR′, —NRNR′R″, —NO2, —NO, —SOR, —SO2R, —CR═CR′R″, —C═CR, -Hal, —N3, —N═NR, —C═NR, —CN, —NC, —NCO, —OCN, —NCS, —SCN, —Si(OR)3, —CNRNR′R″, —NRCNR′NR″R′″, —PO(OR)2, —OPO(OR)2, —SO3R, —OSO3R, -aryl, and/or -heteroaryl, where:
R, R′, R″ and R′″ may independently be hydrogen or C1-C24 alkyl (linear, branched or cyclic),
Hal denotes F, Cl, Br or I;
aryl denotes any aromatic hydrocarbon having 6 to 24 carbon ring atoms which may be monocyclic or annealed (non limiting examples of aryl groups comprise phenyl, naphthyl, azulene, anthracene and/or phenanthrene); and
heteroaryl means any aromatic hydrocarbon having 5 to 24 ring atoms which may be monocyclic or annealed, of which at least one ring atom is a heteroatom selected from the group consisting of N, O, S, and P (non limiting examples of heteroaryl groups comprise pyrrole, indole, imidazole, benzimidazole, pyrazin, pyridazin, triazole, tetrazole, oxazole, isoxazole, thiophene, thiazole, isothiazole, pyridine, chinoline, isochinoline, and/or pyrimidine.)

These functional groups may also comprise cyclic analogues: for example, —OR includes oxtranes, —SR includes thiiranes, —CONRR′ includes lactames and —CO2R includes lactones. The functional groups may also be combined. For example, the combination of a carbonyl group and a heteroaryl group, such as imidazole, leads to an imidazolone.

Most preferred further functional group(s) comprise epoxy, ethyl imidazolidone, hydroxy, N-methylol, carbonyl and/or a further ethylenically unsaturated group which is not conjugated with the other ethylenically unsaturated group.

In a particularly preferred embodiment constituent ‘f’ comprises ethyl imidazolidone methacrylate.

The ethylenically unsaturated compounds for constituent T do not include any of compounds for constituents ‘a’, ‘b’, ‘c’, ‘d’ and/or ‘e’.

Conveniently constituent ‘f’ comprises less than or equal to about 25 wt.-%, more conveniently less than or equal to about 20 wt.-%, most conveniently from about 0.1 to about 10 wt.-%, in particular from about 1.5 to about 2.5 wt-% of the total weight of Polymer P.

Polymer P

In one embodiment of the invention, polymer P comprises
(a) from about 55 to about 80 wt.-% of constituent ‘a’,
(b) from about 8 to about 20 wt.-% of constituent ‘b’,
(c) from about 8 to about 20 wt.-% of constituent ‘c’; and
(d) from about 1 to about 15 wt.-% of constituent ‘d’.

In another embodiment of the invention, polymer P comprises:
(a) from about 60 to about 70 wt.-% of constituent ‘a’,
(b) from about 10 to about 15 wt.-% of constituent ‘b’,
(c) from about 10 to about 15 wt.-% of constituent ‘c’,
(d) from about 1 to about 5 wt.-% of constituent ‘d’,
(e) from about 3.5 to about 5 wt.-% of constituent ‘e’; and
(f) from about 1.5 to about 2.5 wt.-% of constituent ‘f’.

In a still other embodiment of the invention polymer P comprises greater than or equal to about 85 wt.-% of 2-ethylhexyl acrylate, ethyl acrylate and methyl acrylate.

In yet another embodiment of the invention polymer P comprises 2-ethylhexyl acrylate, ethyl acrylate, methyl acrylate and styrene, and optionally (meth)acrylic acid and/or ethyl imidazolidone methacrylate. In a still yet other embodiment polymer P additionally comprises n-butyl acrylate and/or methyl methacrylate.

In a further embodiment of the invention, polymer P comprises
(a) from about 60 to about 70 wt.-% of 2-ethylhexyl acrylate,
(b) from about 10 to about 15 wt.-% of ethyl acrylate,
(c) from about 10 to about 15 wt.-% of methyl acrylate,
(d) from about 1 to about 5 wt.-% of styrene,
(e) from about 0.5 to about 2 wt.-% of acrylic acid and from about 0.5 to about 2 wt.-% of methacrylic acid; and
(f) from about 1 to about 5 wt.-% of ethyl imidazolidone methacrylate, from about 0 to about 1 wt.-% of n-butyl acrylate, from about 0 to about 0.5 wt.-% of n-dodecyl methacrylate and/or from about 0 to about 1 wt.-% methyl methacrylate.

The glass transition temperature (Tg) of a homopolymer can be determined by conventional methods and if nothing else is mentioned in the present application all glass transition temperatures are measured by differential thermal analysis (DTA) or differential scanning calorimetry (DSC) (see ASTM 3418/82, midpoint temperature).

For the homopolymers of numerous monomers the glass transition temperatures Tg are known in the prior art. It is referred to “Polymer Handbook, 2d Ed. By J. Brandrup & E. H. Immergut, 1975, J. Wiley & Sons”. If a certain monomer is not already explicitly classified as one of the constituents in this specification the Tg values disclosed in the above referenced handbook should serve as the decisive basis whether a certain monomer falls under the definition of any of the constituents. Only if a Tg value of a distinct homopolymer is disclosed neither in this specification nor in the above referenced handbook, the Tg value obtained according to DTA or DSC measurements becomes decisive.

The invention also relates to the preparation of aqueous polymer dispersions containing Polymer P.

Polymer P of the invention is preferably obtained by emulsion polymerization. The emulsion polymerization is preferably carried out at a temperature of from about 30° to about 100° C., more preferably from about 50° to about 95° C. The polymerization medium may consist exclusively of water, but also mixtures of solvents which are at least partially soluble in water may be used, e.g. a mixture of methanol and water or a mixture of isopropanol and water. The emulsion polymerization may be carried out in a batch process or alternatively in a semi-continuous process, wherein the reactants and auxiliary additives are added continuously to a reactor in which the polymerization takes place. The reactants may be added as a gradient or stepwise.

In a preferred embodiment of the process according to the invention the polymerization is carried out in a semi-continuous process, wherein initially a relatively small portion of the monomers to be polymerized is fed into the reactor, heated and pre-polymerized. Then, the remainder of the monomers is continuously fed into the reactor, usually via several inlets which are spatially separated from one another. The monomers may be fed into the reactor in pure or in emulsified form (pre-emulsions), as single monomers per inlet or as mixtures of monomers. The mass flow through each inlet may be adjusted individually, i.e. stepwise or as a gradient. The monomers may also be fed into the reactor, thereby establishing a superimposed concentration gradient within the reaction zone of the reactor.

Preferably Polymer P is prepared by emulsion polymerization in water in the presence of suitable surfactants. Preferably the final aqueous polymer dispersion obtained from the polymerization process contains from about 1 to about 5 wt.-% of surfactants (based on the total weight of the monomers to be polymerized). Suitable surface active substances include not only the protective colloids customarily used for carrying out free radical aqueous emulsion polymerizations but also emulsifiers.

Examples of suitable protective colloids are polyvinyl alcohols, cellulose derivatives and vinylpyrrolidone-containing polymers. A detailed description of further suitable protective colloids may be found in Houben-Weyl, Methoden der organischen Chemie, Volume XIV/1, Makromolekulare Stoffe, Georg Thieme Verlag, Stuttgart, 1961, pages 411 to 420.

It is also possible to use mixtures of emulsifiers and/or protective colloids. Preferably, the surface active substances used are exclusively emulsifiers whose relative molecular weights are customarily below 1000, in contrast to protective colloids. They can be anionic, cationic or nonionic in nature. If mixtures of surface active substances are used, the individual components must be compatible with one another, which can be verified in advance of the polymerization by means of a few preliminary experiments if there is any doubt. In general, anionic emulsifiers are compatible with one another and with nonionic emulsifiers. The same is true of cationic emulsifiers, while anionic and cationic emulsifiers are usually incompatible with one another.

According to the invention the emulsifiers are divided into two groups: emulsifiers containing aromatic groups (aromatic emulsifiers) and emulsifiers not containing any aromatic groups (aliphatic emulsifiers). In this specification the simple term “emulsifier” encompasses both, aromatic emulsifiers and aliphatic emulsifiers.

Examples of customary aliphatic emulsifiers are ethoxylated fatty alcohols (such as those with EO degree: 3-50 and C8-C36alkyl), and also alkali metal and ammonium salts of alkyl sulfates (such as C8-C12alkyl), alkali metal and ammonium salts of sulfuric monoesters of ethoxylated alkanols (such as those with EO degree: 4-30 and C12-C18alkyl), alkali metal and ammonium salts of alkylsulfonic acids (such as C12-C18alkyl). Particularly preferred emulisfiers are sodium salts of sulfuric monoesters of ethoxylated alkanols such as following (both available commercially from Cognis under the specified trade names) Disponil FES 32 IS (EO degree: 4, C12-C18alkyl); and Disponil FES 993 IS (EO degree: 12, C12-C18alkyl).

Examples of customary aromatic emulsifiers are ethoxylated mono-, di- and trialkylphenols (EO degree: 3-50, C4-C9alkyl), ethoxylated alkylphenols (EO degree: 3-50, C4-C9alkyl), alkali metal and ammonium salts of alkylarylsulfonic acids (C9-C18alkyl) and sulfonated alkylarylethers.

Further aromatic and aliphatic emulsifiers may be found in Houben-Weyl, Methoden der organischen Chemie, Volume XIV/1, Makromolekulare Stoffe, Georg Thieme Verlag, Stuttgart, 1961, pages 192 to 208.

Preferably the aqueous polymer dispersions according to the invention are the product dispersions which are directly obtained from the emulsion polymerization. These aqueous polymer dispersions may be used as such for different purposes, for example as pressure sensitive adhesives (PSA). PSAs of the invention may be applied to any suitable surface or substrate such as to a polymeric fillm for example used as facestock for labels.

Chain transfer reagents can also be used in the course of the polymerization, for example in amounts of from about 0.01 to about 0.8 parts per weight per 100 parts per weight of the monomers to be polymerized. The function of the chain transfer reagents is to reduce the molar mass of the polymers obtained. Suitable examples are compounds having a thiol group, such as tert-butyl mercaptane, ethylhexyl thioglycolate, mercaptoethanol, mercaptopropyl-trimethoxysilane, n-dodecyl mercaptane or tert-dodecyl mercaptane. According to the invention, the portion of said chain transfer agents can range from about 0.01 to about 0.8 wt.-%, preferably from about 0.05 to about 0.5 wt.-% per 100 parts per weight of the monomers to be polymerized. In case that the final polymer P is devoted for the use as an adhesive for high-gloss film lamination, the use of chain transfer agents is less preferred.

Suitable free radical polymerization initiators are all those which are capable of initiating a free radical aqueous emulsion polymerization. Preference is given to peroxides, with particular preference to alkali metal peroxydisulfates, in particular to ammonium peroxydisulfate. Other suitable initiators are for example redox-initiator systems, such as ascorbic acid/Fe(II)sulfate/sodium peroxydisulfate, tert-butylhydroperoxide/sodium disulfite or tert-butylhydroperoxide/sodium hydroxymethanesulfinic acid. The amount of initiator may generally range from about 0.1 to about 10 wt.-%, preferably from about 0.2 to about 5 wt.-% (based on the total weight of the monomers to be polymerized). It is also possible to use a mixture of different initiators.

The suitable mode of supplying the reaction medium with initiator is known to the skilled person. Before the polymerization starts the initiator may be present in the reactor either in the total amount or only in part. In the latter case, the remainder of the initiator is fed into the reactor via an inlet at a rate dependent on the consumption of the initiator due to the polymerization reaction. In order to avoid that any monomers are still present in the reaction medium after the polymerization has come to an end, generally further initiator is added when the yield of the reaction is in the range of about 95% or above.

Monomers, initiators, chain transfer agents, etc. may be fed into the reactor from the top, at the side or through the bottom of the reactor.

Preferably the polymerization starts with a polymerized seed or with a pre-polymer. The chemical nature of the polymerized seed or the pre-polymer is not limited, as long as the polymers P which are finally obtained from the polymerization process, are covered by the definition of polymers P. Not all of the constituents have to be present simultaneously in the polymerized seed or in the pre-polymer. Preferably the composition of the constituents of the polymerized seed and the composition of the constituents which are polymerized on the polymerized seed differ in at least one constituent. Preferably the polymerized seed comprises n-butyl acrylate which is not present among the constituents to be polymerized on the polymerized seed. Preferably the aqueous dispersion contains as a polymerized seed or a pre-polymer a polymer having a non-zero weight average diameter of from about 10 to about 100 nm, preferably of from about 40 to about 60 nm.

The emulsion polymerization usually yields aqueous polymer dispersions having a solids content of from about 15 to about 75 wt.-%, preferably of from about 40 to about 75 wt.-%. Polymer dispersions having high solids content are preferred.

Preferably, Polymer P obtained and/or obtainable according to the invention has an average particle size of from about 200 to about 250 nm. The pH of the aqueous dispersions is preferably in the range of from about 4 to about 9.

In one embodiment of the invention the polymer is prepared using a first, a second and a third pre-mix tank and a reactor. In a first step of the process the first and the second pre-mix tank are fed with water and emulsifier, optionally with further additives such as sodium carbonate and iso-propanol. The mixture is aged for a certain time, preferably for 15 minutes. Then, the monomers which are to be polymerized are added to the solutions contained in the first and the second pre-mix tank. It is possible that both pre-mix tanks are fed with the same monomers in identical amounts. Alternatively, it is also possible to add different amounts of different monomers to the pre-mix tanks. A chain transfer agent such as n-dodecyl mercaptan is preferably added to the pre-emulsion of the first or the second pre-mix tank. It is also possible, that the same chain transfer agent or a different chain transfer agent is added to the pre-emulsion in the other pre-mix tank. The third pre-mix tank is fed with water and initiator and the reactor is fed with water, then is heated to the desired reaction temperature and fed with initiator and seed. The polymerization is then performed by continuously supplying the reactor with the pre-emulsions from the first and the second pre-mix tank and with the solution of the initiator from the third pre-mix tank. Both pre-emulsions can be fed simultaneously directly in the reactor or one after each other, or the second pre-emulsion fed to the first pre-emulsion and simultaneously to the reactor. Preferably the pre-emulsions are added during a period of some hours, preferably during 2 to 8 hours. When the total initial content of the three pre-mix tanks has been transferred into the reactor the mixture is aged and cooled. Additional amount of initiator can be added to reduce the level of free monomers. Further additives such as biocides can then be added.

In another embodiment of the invention the preparation of the polymers starts from a single mixture containing all monomers to be incorporated into the polymer chain. A fraction of this mixture is fed into the reactor and the polymerization is initiated. If desired, polymerized seed material may be present as well. After initiation of the polymerization the remainder of the monomer mixture is added to the reaction solution stepwise or as a gradient. Simultaneously, a variable amount of the chain transfer agent is added to the reactor, optionally as a gradient. Alternatively, the total amount of the mixture containing all monomers is fed to the reactor before the polymerization is initiated. Then, the chain transfer agent is added to the reactor in various amounts during the polymerization, preferably as a gradient. The continuous addition of chain transfer agent during the polymerization allows control of the distribution of the molecular mass of the polymers.

By varying the concentration of the chain transfer agent in the feeds to the reactor, it is possible to broaden the distribution of molecular masses and to control the particle morphology. This principle is known in the prior art, e.g. it can be referred to U.S. Pat. No. 4,039,500.

Polymer P, particularly aqueous polymer dispersions thereof; may be used as adhesives, preferably as pressure sensitive adhesives. Preferably Polymer P is used in form of aqueous dispersions, which may be used without addition of any further additives. However, it is also possible that further additives, such as tackifiers, defoaming agents, thickening agents, coagulants, softeners, pigments, surfactants, biocides or fillers are added.

Examples of suitable tackifiers are resins such as kolophonium resins (e.g. abientinic acid) and their derivatives, such as esters thereof. Kolophonium esters may be obtained from alcohols such as methanol, ethandiol, diethylenglycol, triethylenglycol, 1,2,3-propanetriol, pentaerythrit, etc. Other suitable tackifiers are cumaron-inden resins, polyterpene resins, hydrocarbon resins based on unsaturated hydrocarbons, such as butadiene, pentene, methylbutene, isoprene, piperylene, divinylmethane, pentadiene, cyclopentene, cyclopentadiene, cyclohexadiene, styrene and styrene derivatives. Further suitable tackifiers are polyacrylates having a relatively low molecular weight (mean average weight generally below 30000). These polyacrylates generally are composed of C1-C8alkyl(meth)acrylates.

Further aspects and preferred embodiments of the invention are given in the claims.

The invention is further illustrated by the following non-limiting examples:

Two polymers, the composition of which is shown in Tables 2a and 3a here below, were prepared according to the invention:

TABLE 2a

| Example 1 | | [g] | [wt. %] |
|---|---|---|---|
| a | 2-ethylhexyl acrylate | 797.98 | 65.27 |
| a | n-butyl acrylate | 9.73 | 0.80 |
| | n-dodecyl mercaptan | 1.20 | 0.10 |
| b | ethyl acrylate | 148.88 | 12.18 |
| c | methyl acrylate | 148.88 | 12.18 |
| d | methyl methacrylate | 4.50 | 0.37 |
| d | Styrene | 33.55 | 2.74 |
| e | Methacrylic acid | 35.74 | 2.92 |
| e | acrylic acid | 12.28 | 1.00 |
| f | ethylimidazolidone | 29.78 | 2.44 |
| | methacrylate | | |
| | Total | 1222.52 | 100.00 |

TABLE 3a

| | Example 2 | [g] | [wt. %] |
|---|---|---|---|
| a | 2-ethylhexyl acrylate | 797.98 | 65.63 |
| a | n-butyl acrylate | 9.73 | 0.80 |
| | n-dodecyl mercaptan | 0.60 | 0.05 |
| b | ethyl acrylate | 148.88 | 12.24 |
| c | methyl acrylate | 148.88 | 12.24 |
| d | methyl methacrylate | 4.50 | 0.37 |
| d | styrene | 33.55 | 2.76 |
| e | methacrylic acid | 35.74 | 2.94 |
| e | acrylic acid | 12.28 | 1.01 |
| f | ethylimidazolidone | 23.82 | 1.96 |
| | methacrylate | | |
| | Total | 1215.96 | 100.00 |

The polymers were polymerized in a reactor at a polymerization temperature of 80° C. in a manner known per se. During the course of the polymerization two pre-emulsions (pre-emulsion 1 and pre-emulsion 2) were fed into the reactor. Pre-emulsion 2 is introduced in pre-emulsion 1 (in pre-mix tank 1) while pre-mix tank 1 content is fed into the reactor. In parallel, the initiator solution (in pre-mix tank 3) is fed into the reactor. The initial composition of the pre-emulsions and the initial reactor content are shown in the Tables 2b and 3b:

TABLE 2b
Example 1

| | | | Weight [g] | |
|---|---|---|---|---|
| | | | Bottom | |
| Steps | Raw Materials | Dilution | tank | Pre-emulsion |
| Reactor | H2O | | 484.68 | |
| | polymerized seed | 37% | 24.32 | |
| Initiator | (NH4)2S2O8 | | 1.20 | |
| | H2O | | 14.40 | |
| Pre-mix tank 3 | (NH4)2S2O8 | | 2.40 | |
| | H2O | | 25.60 | |
| Pre-emulsion 1 | H2O | | | 191.13 |
| In pre-mix | Na2CO3 2H2O | | | 1.20 |
| tank 1 | Disponil FES 993IS | 29% | | 41.38 |
| | Isopropanol | | | 4.50 |
| | 2-ethylhexyl acrylate | | | 10.72 |
| | n-dodecyl mercaptan | | | 1.20 |
| | 2-ethylhexyl acrylate | | | 388.27 |
| | ethyl acrylate | | | 74.44 |
| | methyl acrylate | | | 74.44 |
| | Styrene | | | 11.91 |
| | methacrylic acid | | | 17.87 |
| | acrylic acid | | | 5.96 |
| | ethyl imidazolidone | | | 14.89 |
| | methacrylate | | | |
| Pre-emulsion 2 | H2O | | | 191.13 |
| In pre-mix | Na2CO3 2H2O | | | 1.20 |
| tank 2 | Disponil FES 993IS | 29% | | 41.38 |
| | Isopropanol | | | 4.50 |
| | 2-ethylhexyl acrylate | | | 398.99 |
| | ethyl acrylate | | | 74.44 |
| | methyl acrylate | | | 74.44 |
| | Styrene | | | 11.91 |
| | methacrylic acid | | | 17.87 |
| | acrylic acid | | | 5.96 |
| | ethyl imidazolidone | | | 14.89 |
| | methacrylate | | | |
| Realimentation | Fe(NO3)3 | 10% | 0.05 | |
| | Luperox H70 | 10% | 6.00 | |
| | Rongalit C | 5% | 12.00 | |
| Formulation | Biocide K10 | 1% | 4.50 | |

TABLE 3b
Example 2

| | | | Weight [g] | |
|---|---|---|---|---|
| | | | Bottom | |
| Steps | Raw Materials | Dilution | tank | Pre-emulsion |
| Reactor | H2O | | 484.68 | |
| | polymerized seed | 37% | 24.32 | |
| Initiator | (NH4)2S2O8 | | 1.20 | |
| | H2O | | 14.40 | |
| Pre-mix tank 3 | (NH4)2S2O8 | | 2.40 | |
| | H2O | | 25.60 | |
| Pre-emulsion 1 | H2O | | | 188.70 |
| Pre-mix tank 1 | Na2CO3 2H2O | | | 1.20 |
| | Disponil FES 993IS | 29% | | 41.38 |
| | Isopropanol | | | 4.50 |
| | 2-ethylhexyl acrylate | | | 10.72 |
| | n-dodecyl mercaptan | | | 0.60 |
| | 2-ethylhexyl acrylate | | | 388.27 |
| | ethyl acrylate | | | 74.44 |
| | methyl acrylate | | | 74.44 |
| | styrene | | | 11.91 |
| | methacrylic acid | | | 17.87 |
| | acrylic acid | | | 5.96 |
| | ethyl imidazolidone | | | 11.91 |
| | methacrylate | | | |
| Pre-emulsion 2 | H2O | | | 188.70 |
| Pre-mix tank 2 | Na2CO3 2H2O | | | 1.20 |
| | Disponil FES 993IS | 29% | | 41.38 |
| | Isopropanol | | | 4.50 |
| | 2-ethylhexyl acrylate | | | 398.99 |
| | ethyl acrylate | | | 74.44 |
| | methyl acrylate | | | 74.44 |
| | styrene | | | 11.91 |
| | methacrylic acid | | | 17.87 |
| | acrylic acid | | | 5.96 |
| | ethyl imidazolidone | | | 11.91 |
| | methacrylate | | | |
| Realimentation | Fe(NO3)3 | 10% | 0.05 | |
| | Luperox H70 | 10% | 6.00 | |
| | Rongalit C | 5% | 12.00 | |
| Formulation | Biocide K10 | 1% | 4.50 | |

In Tables 2b and 3b:
Luperox H70 is tert.-butylhydroperoxyde 70% with 30% water;
Rongalit C is sodium hydroxymethanesulphinate
Biocide K10 is a biocide from Thor. It is an aqueous mixture of 5-chloro-2-methyl-4-isothiazolin-3-one and 2-methyl-4-isothiazoline-3-one

Example 2 described in Table 3b is treated in the same manner as Example 1 given in Table 2b.

The polymerized seed used in these syntheses is based on the following monomer composition: methyl methacrylate (18.5 wt.-%), styrene (40 wt.-%), n-butyl acrylate (40 wt.-%) and acrylic acid (1.5 wt.-%/o):

TABLE 4

| polymerized seed | | quant. [g] | quant. [%] |
|---|---|---|---|
| Reactor | demineralized water | 1323.00 | 47.432 |
| | Disponil FES 32 IS | 83.33 | 2.988 |
| Initiation | (NH4)2S2O8 | 2.00 | 0.072 |
| pre-emulsion | demineralized water | 345.61 | 12.391 |
| | Disponil FES 32 IS | 16.70 | 0.599 |
| Monomers | methyl methacrylate | 185.00 | 6.633 |
| | Styrene | 400.00 | 14.341 |
| | n-butyl acrylate | 400.00 | 14.341 |
| | acrylic acid | 15.00 | 0.538 |
| Formulation | ammonia 25% | 13.00 | 0.466 |
| | Acticide AS | 2.80 | 0.100 |
| | Acticide MV | 2.80 | 0.100 |

| | | |
|---|---|---|
| Total | 2789.24 | 100.000 |
| % dry material | 37.14 | |

Acticide AS and Acticide MV are biocides from Thor.

Acticide AS is a water based formulation of 1,2-benzisothiazoline-3-one and a thiazole derivative

Acticide MV is a formulation of 5-chloro-2-methyl-4-isothiazolin-3-one (CIT) and 2-methyl-4-isothiazolin-3-one (MIT)

For making the polymerised seed, the reactor is fed with water and surfactant, then heated to 80° C. Then, initiator is introduced and polymerization started by feeding monomer pre-emulsion in 3-4 hours. The mixture is aged for 1 hour then cooled. The adhesive properties of the aqueous polymer dispersions obtained from composition 1 and composition 2 were tested according to standard test methods:

Test Methods

Peel adhesion (peel strength), resistance to shear from a standard surface (shear strength) and “Quick-Stick” tack (loop tack strength) were determined according to FINAT test methods (FTM) Nos. 1, 8 and 9. These standard test methods FTM 1, FTM 8 and FTM 9 are described in FINAT Technical Handbook 5th edition, 1999, (published by FINAT, P.O. Box 85612 NL-2508 CH The Hague, The Netherlands).

a) Preparing the Test Strips

The test dispersion was mixed with a wetting agent (Servoxyl VIA 70: sulfoccinate-type surfactant (35 wt % in water)). The mixture proportion was 1.5 g Servoxyl in 100 g latex. Then, it was applied to siliconized paper in a thin film, using a bar coater, and was dried at 110° C. for 3 minutes. The gap height of the coating bar was chosen so that the weight per unit area of the dried adhesives was 20-22 g/m2. A commercially customary plasticized PVC film (80 μm thick) was placed on the dried adhesive and rolled on firmly using a manual roll. The resulting adhesive was stored under atmospheric conditions for at least 24 h, then it was cut into 25 mm wide strips. Their minimum length was 175 mm.

b) Testing the Shear Strength as a Measure of Cohesion (According to FINAT FTM 8)

After the sfficonized paper had been peeled off, the film test strip was bonded to the edge of a stainless steel sheet so as to give a bond area of 25 mm×25 mm. 10 minutes after bonding, a 1000 g weight was fastened to the protruding end of the film strip, and the metal sheet was suspended vertically (ambient conditions: 23° C., 50% relative humidity). The shear strength was taken as the time in minutes, until the adhesive bond failed under the influence of the weight, as the mean from the results of three test specimens.

c) Testing the Peel Strength as a Measure of Adhesion (According to FINAT FTM 1)

After the siliconized paper had been peeled off, the film test strip was bonded to a glass plate (ambient conditions: 23° C., 50% relative humidity). The test strip was leaved for 20 min, then the strip was peeled off at an angle of 180° and at a rate of 300 mm/min with the aid of a tensile testing apparatus. The peel strength was taken as the force, in N/25 mm, required to do this, again as a mean from the results of three test specimens. The procedure was repeated with a second set of strips with a period of 24 h before testing.

d) Testing the Loop Tack Strength as a Measure of Tackiness (According to FINAT FTM 9)

After the siliconized paper had been peeled off, the two ends of the adhesive coated facing material strip were hold together and a loop was formed from the strip, adhesive surface outermost, by bringing the two ends together. The loop ends were clamped for a distance of 10 mm into the top jaw of the tensile tester leaving the loop hanging vertically downwards. The loop was brought into contact with a glass plate at a speed of 300 mm/minute. When full contact over the glass plate had been achieved (25 mm×25 mm), the direction of the machine was reversed and separation allowed to take place at a speed of 300 mm/minute (ambient conditions: 23° C., 50% relative humidity). The loop tack strength was taken as the force, in N/25 mm, required to do this, again as a mean from the results of three test specimens.

e) Testing the Loss of Transparency as a Measure of Water-Whitening Resistance

This method covers the measurement of the loss of transparency of an adhesive coated PVC film after immersion for 7 and 60 minutes in a deionized water bath at 200 to 23° C. (Reference: ASTM Method D 1746-70)

The following devices were used for the determination of the water whitening:
HAZE-GUARD PLUS (from Byk-Gardner)
Commercial PVC transparent film
Standard commercial siliconized paper
Meyer bar coater
Drying oven (110° C.)
Finat roller of 2 kg
   Preparation of the Formulation

To 200 g of composition 1 and composition 2, respectively were added under slow agitation 3 g of Servoxyl VLA 2170 35%. Under slow agitation NH4OH 25% was added to adjust the pH to 6-6.5. The agitation was continued for further 30 minutes and the formulation was allowed to stand overnight.

Preparation of the Coated Material

An adhesive film of 20±g/m2 on silicone paper with an appropriate Meyer bar coater was drawn. The film was dried for 3 minutes at 110° C. in the drying oven without a glass plate. The film was covered with PVC films. Thereafter, it was rolled over the assembly with the Finat roller and the weight/m2 of dry adhesive was controlled as follows:

Samples Measurements

After calibration of the HAZE-GUARD PLUS, the loss in total transmittance light through the coated material was measured under the following conditions:

After the siliconized paper had been peeled off, a 25×25 mm sample cut from coated material was immersed in a detonized water bath at 23° C. for 7 minutes. Then the total transmittance of light trough the strip was measured using a haze-guard plus (from BYK Gardner) (A) and compared to the total transmittance of the strip before immersion (3). The water-whitening was taken, in %, as the transmittance difference (A)-(B), again as a mean from the results of three test specimens. The procedure was repeated with a second set of strips with a period of 60 minutes in immersion before testing.

TABLE 5

| | Composition 1 | Composition 2 |
|---|---|---|
| solid content [wt. %] 1 | 54.75 | 54.55 |
| Brookfield viscosity 50 rpm (cps) 1 | 148 | 140 |
| pH1 | 4.8 | 4.7 |
| particle size [nm] 1,2 | 219 | 221 |
| shear strength [min] | >10000 | >10000 |
| peel strength 20 min [N/25 mm] | 15.5 | 12.3 |
| peel strength 24 h [N/25 mm] | 15.1 | 14.5 |
| loop tack [N/25 mm] | 13.7 | 13.9 |
| water-whitening 7 min [%] | 0.3 | 1.2 |
| peel strength 20 min [N/25 mm] after | 8.8 | 8.2 |
| ageing 7 days at 60° C.3 | | |
| peel strength 24 h [N/25 mm] after | 10.3 | 9.5 |
| ageing 7 days at 60° C.3 | | |
| loop tack [N/25 mm] after ageing | 11.5 | 10.1 |
| 7 days at 60° C.3 | | |
| water whitening 60 min [%] | 2.2 | 4.3 |

1 Mean value from results of three measurements
2 Weight-averaged values determined by dynamic light scattering (Nicomp 370, Particle Sizing Systems).
3 The testing strips are aged for 7 days at 60° C. in an oven between 2 glass plates.
   Then the normal procedure to measure peel strength is followed as described earlier.

As illustrated by the above examples, the aqueous polymer dispersions according to the invention exhibit low water whitening characteristics and have a high shear strength together with excellent peel strength and loop tack.

1. A polymer which comprises, optionally consists essentially of
(a) from about 5 to about 92.5 wt.-% of at least one alkyl acrylate, the homopolymers of which have a Tg of less than or equal to about −40° C. (constituent a);
(b) from about 2.5 to about 30 wt.-% of at least one alkyl (meth)acrylate, the homopolymers of which have a Tg of from about −25° C. to about 0° C. (constituent b);
(c) from about 2.5 to about 30 wt.-% of at least one alkyl (meth)acrylate, the homopolymers of which have a Tg of from about 0° C. to about 20° C. (constituent c);
(d) from about 1 to about 30 wt.-% of at least one ethylenically unsaturated monomer, the homopolymers of which have a Tg of greater than or equal to about 20° C., and which do not contain a functional group selected from hydroxyl, acid, acid anhydride, nitro, epoxy and amino groups (constituent d);
(e) from about 0 to about 10 wt.-% of at least one ethylenically unsaturated monomer having at least one acid group or acid anhydride group (constituent e); and
(f) from about 0 to about 60 wt.-% of at least one ethylenically unsaturated monomer having either no further functional group or in addition to the ethylenically unsaturated group at least one functional group other than an acid or an acid anhydride group (constituent f).

2. The polymer according to claim 1, where constituent ‘a’ comprises at least one alkyl acrylate selected from the group consisting of n-butyl acrylate, 2-ethylbutyl acrylate, hexyl acrylate, 2-ethylhexyl acrylate, nonyl acrylate and octyl acrylate.

3. The polymer according to claim 1, where constituent ‘b’ comprises at least one alkyl (meth)acrylate selected from the group consisting of ethyl acrylate, sec-butyl acrylate, dodecyl acrylate, isobutyl acrylate and isopropyl acrylate.

4. The polymer according to claim 1, where constituent ‘c’ comprises at least one alkyl (meth)acrylate selected from the group consisting of methyl acrylate and n-butyl methacrylate.

5. The polymer according to claim 1, where constituent ‘d’ comprises a monomer which is selected from the group consisting of: (i) alkyl (meth)acrylates, (ii) vinylaromatic compounds, (iii) vinylhalides, (iv) vinylnitriles and (v) vinylesters of carboxylic acids.

6. The polymer according to claim 5, where constituent ‘d’ comprises at least one ethylenically unsaturated monomer selected from the group consisting of methyl methacrylate, tert-butyl acrylate, methylstyrene, para-methylstyrene, tert-butylstyrene, styrene, vinyl chloride, methacrylonitrile, vinyl acetate and vinyl formate.

7. The polymer according to claim 1, where constituent ‘e’ comprises at least one ethylenically unsaturated monomer selected from the group consisting of (meth)acrylic acid, maleic acid, maleic acid anhydride, maleic acid monoester and fumaric acid monoester.

8. The polymer according to claim 1, where constituent ‘f’ comprises at least one ethylenically unsaturated compound having a group selected from the groups consisting of epoxy groups, hydroxyl groups, ethyl imidazolidone groups, N-methylol groups, carbonyl groups or further ethylenically unsaturated groups which are not conjugated with the other ethylenically unsaturated group.

9. The polymer according to claim 1, where greater than or equal to about 85 wt.-% of the polymer comprises 2-ethylhexyl acrylate, ethyl acrylate and methyl acrylate.

10. The polymer according to claim 1, which comprises 2-ethylhexyl acrylate, ethyl acrylate, methyl acrylate and styrene, and optionally (meth)acrylic acid, ethyl imidazolidone methacrylate, n-butyl acrylate, n-dodecyl methacrylate and/or methyl methacrylate.

11. The polymer according to claim 10, which comprises:
(a) from about 60 to 70 wt.-% of 2-ethylhexyl acrylate,
(b) from about 10 to about 15 wt.-% of ethyl acrylate,
(c) from about 10 to about 15 wt.-% of methyl acrylate,
(d) from about 1 to about 5 wt.-% of styrene, 30
(e) from about 0.5 to about 2 wt.-% of acrylic acid and from about 0.5 to about 2 wt.-% of methacrylic acid, and
(f) from about 1 to about 5 wt.-% of ethyl imidazolidone methacrylate, from about 0 to about 1 wt.-% of n-butyl acrylate, from about 0 to about 0.5 wt.-% of n-dodecyl methacrylate and/or from about 0 to about 1 wt.-% methyl methacrylate.

12. An aqueous polymer dispersion comprising a polymer according to claim 1 which is dispersed in an aqueous medium.

13. The dispersion according to claim 12, which comprises at least one aliphatic emulsifier.

14. The dispersion according to claim 12, which comprises at least one aliphatic emulsifier and one aromatic emulsifier.

15. (canceled)

16. (canceled)

17. Process for preparing a polymer according to claim 1 by contacting the constituents ‘a’, ‘b’, ‘c’, and ‘d’ and optionally ‘e’ and/or ‘f’ in form of the monomers with a suitable polymerization initiator in a reaction medium.

18. Process according to claim 17, where during polymerization a chain transfer agent is gradually added to the reaction medium.

19. Process according to either claim 17, where a polymerized seed is initially added to the reaction medium whereby the composition of the polymerized seed differs in at least one constituent from the composition of the monomers in the reaction medium.

20. Process according to claim 19, where the polymerized seed contains n-butyl acrylate and the monomers in the reaction medium do not contain n-butyl acrylate.

21. Polymer according to claim 1, which is obtained and/or obtainable from a process according to which comprises by contacting the constituents ‘a’, ‘b’, ‘c’, and ‘d’ and optionally ‘e’ and/or ‘f’, in form of the monomers with a suitable polymerization initiator in a reaction medium.
